(19) United States
(12) Patent Application Publication
(10) Pub. No.: US 2002/0048817 A1
(43) Pub. Date: Apr. 25, 2002

(54) Composition and process for fabrication of absorbance and fluorescence standards

(75) Inventors: Ahmad Yekta, St. Catharines (CA); Zahra Masoumi, St. Catharines (CA)

Correspondence Address: DARBY & DARBY P.C., 805 Third Avenue, New York, NY, 10022, US

(73) Assignee: Imaging Research Inc.

(21) Appl. No.: 09/943,369

(22) Filed: Aug. 29, 2001

Related U.S. Application Data
(60) Provisional application No. 60/229,152, filed on Aug. 30, 2000.

(51) Int. Cl. G01N 31/00; G01N 33/566

(52) U.S. Cl. 436/8; 436/501; 436/19

(57) ABSTRACT

Process and formulation are described allowing fabrication of absorbance and fluorescence standards in cuvettes and micro-well plate, and other desirable containers, with particular application to drug discovery and high throughput screening of bioactive systems. The material medium is capable of incorporating a large number of dyes, individually or in combination, and can closely mimic real aqueous assays in optical properties such as the dye spectra, transparency, refractive index, and shapes of meniscus. The medium is compatible with addition of formulation components for control of foaming, vapor pressure, freezing point, dye bleaching, and molecular rotational correlation times. The process starts with the dispensing of a fluid dye-containing liquid into the vessel of choice, and its subsequent viscosification by chemical or physical means into a viscous gel. After further processing for stability, the container can be sealed with appropriate means. The standards are useful for calibration of spectrophotometric or fluorometric plate readers and imagers for correction of systematic spatial errors, for calibration of absolute intensities, and their replicates may be used to allow cross comparison of different instruments. Specialized standards can be used to check instrument performance in specialized assays relying on fluorescence resonance energy transfer (FRET), time-resolved fluorescence (TRF) and fluorescence polarization (FP).

This application claims the benefit of U.S. Provisional Application No. 60/229,152, filed Aug. 30, 2000, which is herein incorporated by reference.

### FIELD OF THE INVENTION

This invention relates to a standard for calibrating an instrument, such as a spectrometer (e.g., a fluorometer or a spectrofluorometer), a multi-well plate reader, or an imager, comprising one or more viscosity changing polymers and at least one dye, methods of preparing the same, and methods for calibrating spectrometers with the same.

### BACKGROUND OF THE INVENTION

The invention described herein relates to composition and process for fabrication of absorbance and fluorescent reference materials in formats such as cuvettes and micro-well plates. The intended utility of the standards includes calibration of spectrophotometers, fluorometers, fluorescent plate readers and imagers. Although other dye concentration ranges are not ruled out, in general fluorescent standards contain dye concentrations at about 0.1 μM or less, while absorbance standards contain the dyes at higher than 1 μM. This application, for the sake of brevity, only refers to fluorescent standards and not absorbance standards as the methods and procedures of fabrication are nearly identical.

Solutions of many dye molecules, when illuminated by visible or ultraviolet (UV) light, emit back a fraction of the absorbed energy as fluorescent light of longer wavelength. The fluorescence signal maybe used to obtain information about the dye and/or other reagents influencing it. Three aspects of the technique of fluorometry make it an especially powerful tool: (a) it is extremely sensitive allowing measurements on very small quantities; (b) it has special application to assaying of many biological systems, even when the analyte of interest does not fluoresce, because one may tag the bioactive compound with a highly fluorescent molecule; and (c) numerous fluorescent probes are available commercially (Haugland, RP, in Spence MDZ Ed., Handbook of Fluorescent Probes and Research Chemicals, Molecular Probes, Inc., Eugene, Oreg., 1996).

Fluorometers have three principal components: (a) a light source for excitation; (b) one or more filters and/or dispersive monochrometers for selecting wavelength regions of interest; and (c) a detector which converts the impinging fluorescence to an electrical signal. Depending on sensitivity and cost requirements, the detector may be a diode, charge-coupled device (CCD), or photomultiplier tube (PMT). Most traditional fluorometers are diode- or PMT-based and measure on a single sample at a time. More recent imaging instruments use a CCD to simultaneously image and quantify many fluorescing samples at once (Ramm, P, “Imaging Systems in Assay Screening,” Drug Discovery Today, 4, 401-410 (1999)).

PMT's and CCD's can be very responsive to exceedingly low levels of light, placing fluorometry among the most sensitive of all analytical tools. Sensitivity is particularly important to biological assays because of the scarcity and high cost of bioactive compounds. In the pharmaceutical industry, fluorometry is applied to high throughput screening (HTS), where drug candidate compound libraries are screened at rates exceeding 100,000 samples per day, each in minute quantities (Pope, AJ; Haupts, UM; Moore, KJ, “Homogenous Fluorescence Readouts for Miniaturized High-Throughput Screening: Theory and Practice,” Drug Discovery Today, 4, 350-362 (1999)). In this methodology, every candidate is placed within a small cavity (well) of a micro-well plate. Plates are formatted to contain numerous wells, e.g., 96, 384, or 1536 wells. Other reagents are added into each well, bringing the corresponding total volume of the assay in the range of 100 to 10, down to about 1 microliter per well, respectively.

In HTS two types of fluorometers are in common use. The first type is PMT-based plate scanners, in which the plate moves, one well at a time, under an illumination/detection construct. In these instruments all wells are supposed to be measured in an identical manner. In fact, there is no assurance of this and one needs a uniformly dispensed sample plate to check that all wells produce the same signal. The second type is CCD-based imaging systems, which image the whole plate at once, allowing much higher throughput. In these instruments the efficiencies of illumination and fluorescent light collection depend on the well position, and one needs to calibrate to correct for systematic spatial reading errors.

There are many reasons why users may wish to calibrate their instruments. First is an interest in instrument reproducibility over time. Second, calibration can correct systematic instrumental errors so results can be compared across different instruments and/or laboratories. Third, calibration can allow conversion of the raw units of measured signals, always electrical in nature, into absolute units expressed as analyte quantities, such as concentration, number of molecules, etc. Additionally, calibration of plate imagers and readers can correct position-dependent systematic errors.

Calibrations are performed by making measurements on fluorescence standards. A standard is a properly characterized source of signal, the replicates of which can be tested reproducibly as references across different laboratories. Calibrations can be categorized into two classes, namely, those correcting for spectral measurements and those correcting for intensity measurements.

Spectral calibrations are needed for correcting the readings of spectrometers. Filter fluorometers are free of this requirement because they determine signal intensities at a fixed wavelength, rather than spectral distributions. Such calibrations, once established, are usually stable for about a year or more. More importantly, because a sample's spectral features are highly insensitive to its shape, container format, or the geometry of the experiment, standards for spectral calibrations need not be identical in these respects to those of the samples under analysis. Spectral calibrations are performed to correct instrumental inaccuracy in the reading of wavelengths and nonuniform spectral responses. Inaccuracy in the reading of wavelengths is important to correct only when samples have sharp spectral features with bandwidth less than 5 mn. In this case one usually uses as a standard a low-pressure discharge source which has well-known line spectra (see also ASTM E388-72 (1988)). For example, for higher resolution, one can use low-pressure Hg(Ar) pen lamp standards, such as those available from Oriel Instruments of Stratford, Conn. (http://www.oriel.com/); and for lower resolution, one can use solid standards such as those provided by Photon Technology International, Inc., Lawrenceville, N.J. (http://www.pti-nj.com). Nonuniform spectral response is important to correct when one is interested to obtain true sample emission spectra freed from instrumental distortions. Calibrations of this type require standard sources which have well characterized broad-band emission spectra. For example, one can use calibrated QTH lamps; black bodies, such as those available from Oriel Instruments of Stratford, Conn.; solid-state NIST secondary standards SRM-1931; or fluorescence from freshly prepared solutions of secondary standards, such as quinine bisulfate (See, for example, Parker Calif., “Photoluminescence of Solutions,” Elsevier Publishing Co., New York, 1968; Thompson A, and Eckerle K L, “Standards for Corrected Fluorescence Spectra,” Proc. SPIE, Fluorescence Detection III1054,20-25 (1989), and the references therein; and Gardecki J. A. and Marconcelli M., “Set of Seconard Emission Standards for Calibration of the Spectral Responsivity in Emission Spectroscopy,” Applied Spectroscopy 52:1179-1189 (1998)).

Intensity calibrations are more problematic to establish. Unlike spectral calibrations, they remain valid for short durations only. The instability is related to the fact that the instrumental parameters that control signal strengths are themselves highly variable over time. For example, at any given wavelength, the intensity of light sources and/or the response of detectors fluctuate and drift, while efficiencies of the optical components usually drift, in part due to gradual deposition of contaminants and/or ongoing surface photo-damage. Other sources of difficulty relate to the fact that a fluorescent analyte's signal strength is strongly dependent on characteristics such as the sample's medium, shape, container format, as well as the optical geometry involved in its illumination and fluorescence collection. As a result, an intensity standard has to meet the stringent requirements of mimicking the analyte's aforementioned features before it can be reliably used for instrumental calibration.

Several types of intensity standards can be envisioned that calibrate for: (a) instrumental instabilities over time, from days, to months and longer, so that except for random noise, identical samples result in identical determinations at different times; (b) transformation of an analyte's raw signal into an absolute knowledge of its quantity; and (c) multi-well plate readers' and imagers' systematic position-dependent errors so that, except for random noise, identical samples in different wells result in identical determinations.

Standards may also be categorized into two classes, namely primary or ideal standards and secondary standards. A primary or ideal standard is essentially identical to the analyte sample, except that it contains a known amount of the active compound. Primary standards are the most reliable, but are inherently unstable and need to be prepared afresh for each calibration. A secondary standard is composed of a material that closely mimics the characteristics of a primary standard, but exhibits long term stability, so that it may be used repeatedly. Additionally, secondary standards used for interlaboratory comparisons must have low variances (i.e. each standard is substantially identical to other standards of the same type).

The short-term validity of intensity calibrations has resulted in increased demand for appropriate secondary standards. However, few fluorescence intensity secondary standards are commercially available and are not reliable. The scarcity of these standards is due to the difficulty of their fabrication considering the assortment of dyes involved and the scores of shapes and formats that are in demand. As a result, users have had to resort to in-house preparation of their own primary standards, a time consuming and expensive activity, particularly for assaying of bioactive compounds.

The secondary standards that are currently commercially available are all solid-state, presumably because solidity confers long-term durability. For example, Hitachi Instruments of San Jose, Calif. (http://www.hii.hitachi.com/) markets secondary standards in the form of cuvette-shaped pieces of dye-containing plastic; Labsphere, Inc. of North Sutton, NH (http://www.labsphere.com/) markets a fluorescent whitening agent molded in an acrylic plastic or as inorganic fluors in a specialty plastic; Turner Designs of Sunnyvale, Calif. (http://www.turnerdesigns.corn/) employs Bicron Corp.'s fluorescent fibers (see http://www.bicron.com/fibers.htm) in a special apparatus with adjustable slits, disclosed in International Patent Publication No. WO 00/17627; Precision Dynamics Corp. of San Fernando, Calif. (http://www.pdcorp.corn) markets a dye-impregnated sheet of inorganic material at the bottom of multi-well plates; Pal-Med Inc. of Valhalla, N.Y. markets a dye containing piece of plastic shaped to fit into an individual well of multi-well plates.

At the present time, there are very few commercial products for converting signal intensities into absolute dye quantities. For those working with cuvette-shaped samples, the Hitachi standards would approximate the shape and format, and if the spectra match one's sample, then the values read need be recalibrated in house. Turner Designs' standards are potentially useful for checking of instrumental instabilities over time. Yet the claim of the standards being useful for other types of calibration is not expected to be reliable because of their unusual shapes, formats and optical characteristics, when compared to samples encountered in fluorometry, particularly in multi-well plates.

For users interested in correction of the well-position-dependent errors of plate readers and imagers, Precision Dynamics Corp.'s standard plates promise utility. However, reliability is not assured because real liquid-based assay samples, in addition to having different spectral characteristics, occupy a volume and shape which is quite different from the dye-impregnated sheet at the bottom of wells, as provided by Precision Dynamics Corp. The other alternative, Pal-Med Inc.'s single well plastic filling, would be closer to the sample shape format, but is expensive and time consuming to carry out for hundreds of wells separately.

As an example of the desirable features of a fluorescent micro-well plate secondary standard that this invention claims to make possible, we consider the instance of a real assay that relies on top-read measurements of 10 nM aqueous fluorescein, in black solid-bottom 384-well Costar™ plates (Coming Inc. Life Sciences, Acton, Mass.), at 40 microliter per well, pH 8. The desirable secondary standard should be in the same plate format, with each well uniformly containing a dye with spectral characteristics very close to that of sodium fluorescein in water, with a volume close to 40 microliter per well, and a concave meniscus similar to that found in the real assay. Unlike the fluid assay solution, however, it should be mechanically stable so that it does not change shape, and long-term stable for repeated usage. The amount of the dye in this standard should be such that the resulting signal is equivalent to a primary standard, at 10 nM sodium fluorescein (pH 8), and under identical optical geometries of measurement. With these features, the standard can then be used to calibrate plate imagers and readers for systematic errors of reading at each well position, such that a uniformly dispensed assay plate, except for the random noice of measurement, would results in identical calibrated determinations from all wells. Because the standard mimics a primary standard, its replicates may be used to compare results across different laboratories and or instruments. Clearly, the results would be less accurate if this standard were to be used to calibrate readings on other plate formats or dyes. Consequently, a desirable fabrication process should make possible simultaneous production of different standards, in a variety of plate formats, and with different dyes, as this invention claims.

To meet the foregoing needs the subject invention has been developed for fabrication of stable fluorescence and absorbance standards, closely mimicking spectral and shape formats of various fluorescent test samples, in micro-well plates, cuvettes, or other containers. The invention describes how appropriate processing steps may be used along with novel formulations of commercially available materials to fabricate mechanically and chemically stable media for fluorescent dyes. The resulting standards may be used for absolute intensity calibrations of various fluorometers and plate readers, as well as spectral calibrations of the response of spectrofluorimeters. When the dye concentration is taken sufficiently high, such that optical absorbance is in the range of about 0.1 to 1.0, the plate or cuvette may be used as an absorbance standard, for calibration of spectrophotometers, absorbence-reading multi-well plate readers, and imagers.

The key criteria behind the invention are the following: (1) The dye-containing medium should closely mimic the optical properties of the aqueous assay of interest: e.g., transparency, refractive index, shape of meniscus, and the hydrogen bonding of the dye which influences its spectral characteristics; (2) The medium should solubilize both hydrophilic and hydrophobic dyes; (3) The medium should be compatible with addition of other formulation components for control of foaming, vapor pressure, freezing point, dye bleaching, and molecular rotational correlation times; (4) At the dispensing stage the medium should be sufficiently fluid to allow ease of delivery into various containers such as microwells, cuvettes, or other desired vessels; (5) After dispensing, a processing step should trigger a large viscosity increase in the formulation, while preserving the integrity of its shape and volume. Viscosity should be high enough so that the content of an inverted vessel, on its own, would not pour out, or change shape; (6) the medium should be chemically and mechanically stable in the long term.

### SUMMARY OF THE INVENTION

The present invention is a standard for calibrating an instrument, such as a spectrometer (e.g., a fluorometer or a spectrofluorometer), a multi-well plate reader, or an imager, comprising one or more viscosity changing polymers and at least one dye. The viscosity of the viscosity changing polymer in the standard is preferably at least about 10,000 cP and more preferably at least about 100,000 cP. A preferred type of viscosity changing polymer is a pH responsive polymer. According to one embodiment, the dye is a fluorescent dye. The standard may be incorporated into a container, such as a plate, cuvette, or one or more micro-wells. The standard of the present invention is easy to prepare, can include hydrophobic and hydrophilic dyes, and can mimic a variety of assays. For example, the degree of fluorescence polarization and fluorescence resonance energy transfer of the standard can be adjusted. As a result, it is particularly useful for calibrating instruments involved in high throughput screening.

Another embodiment is a method of preparing the standard of the present invention. The method includes mixing a viscosity changing polymer in liquid form with at least one dye and gelling the resulting mixture. The viscosity change may be triggered by a physical (e.g., temperature change) or chemical (e.g., pH change) transformation.

Yet another embodiment is a method of preparing the standard of the present invention in a container. The method includes dispensing a viscosity changing polymer and at least one dye in liquid form into a container and gelling the mixture. Preferably, the viscosity changing polymer and dye are mixed prior to being dispensed into the container. Since the viscosity changing polymer is initially fluid, it can easily be dispensed into the container. Once it gels, the standard is stable and not movable.

Yet another embodiment is a method for calibrating an instrument, such as a spectrometer (e.g., a fluorometer or a spectrofluorometer), a multi-well plate reader, or an imager. The method includes calibrating the instrument with the standard of the present invention.

### BRIEF DESCRIPTION OF THE DRAWINGS

FIG. 1 depicts the chemical structure of an exemplary HASE polymer disclosed by Jenkins et al., Influence of Alkali-Soluble Associative Emulsion Polymer Architecture on Rheology,”Chapter 23 in J. E. Glass Ed., Advances in Chemistry Series 248, Hydrophilic Polymers, Performance with Environmental Acceptability, ACS, Washington, D.C., 1996, pp. 425-447.

### DETAILED DESCRIPTION OF THE INVENTION

The standard of the present invention includes one or more viscosity changing polymers and at least one dye. The standard may be incorporated into a container, such as a plate, cuvette, or one or more micro-wells.

The term “viscosity changing polymer” refers to an aqueous polymer solution in which its viscosity varies with a physical (e.g., temperature) or chemical (e.g., pH) change. Preferably, the viscosity changing polymer can exist in fluid (e.g., liquid) and viscous (e.g., gel) states. Examples of viscosity changing polymers include, but are not limited to, pH responsive polymers, temperature responsive polymers, and mixtures thereof. The viscosity of the fluid state of the viscosity changing polymer preferably can range from about 1 to about 1,000 cP and more preferably from about 1 to about 100 cP. The viscosity of the viscosity changing polymer in the standard (e.g., gel state) is preferably at least about 10,000 cP and more preferably at least about 100,000 cP.

The terms “pH responsive polymer” and “temperature responsive polymer” are defined herein as a polymer which increases in viscosity as its pH changes (e.g., increases) or its temperature decreases, respectively.

According to one preferred embodiment, the viscosity of the pH responsive polymer increases, preferably irrversibly, at a pH of about 5 or higher and the polymer is a liquid at a pH of about 4.5 or lower. The pH of the polymer can be adjusted by addition of a base, such as ammonia, an amine, or a non-volatile inorganic base, such as sodium hydroxide, potassium carbonate, or the like. Preferably, the pH responsive polymer becomes translucent or transparent to light in the desired wavelength range as the pH changes (i.e., as the viscosity of the pH responsive polymer increases). According to one embodiment, the pH responsive polymer becomes transparent to light at a wavelength of from about 300 to about 1,000 mn when the polymer gels.

Preferred pH responsive polymers include, but are not limited to, hydrophobically-modified alkali-swellable emulsions (HASE), such as acrylic carboxylate emulsion polymers and alkali-swellable emulsion urethane-modified emulsion polymers. Suitable pH responsive polymers include, but are not limited to, those described in U.S. Pat. Nos. 4,384,096; Re. 33,156; 5,292,843; 5,461,100; 5,681,882; 5,770,760; 5,874,495; and 5,916,967 and Wetzel et al., “Associative Thickeners,” Chapter 10 in J. E. Glass Ed., Advances in Chemistry Series 248, HydrophilicPolymers, Performance with Environmental Acceptability, ACS, Washington, D.C., 1996, pp. 163-179 and Jenkins et al., Influence of Alkali-Soluble Associative Emulsion Polymer Architecture on Rheology,” Chapter 23 in J. E. Glass Ed., Advances in Chemistry Series 248, Hydrophilic Polymers, Performance with Environmental Acceptability, ACS, Washington, D.C., 1996, pp. 425-447, all of which are hereby incorporated by reference. Preferred alkali-swellable emulsion urethane-modified emulsion polymers include UCAR® Polyphobe® rheology modifiers sold by Dow Chemical Co. of Midland, Mich., such as UCAR® Polyphobe® TR-116.

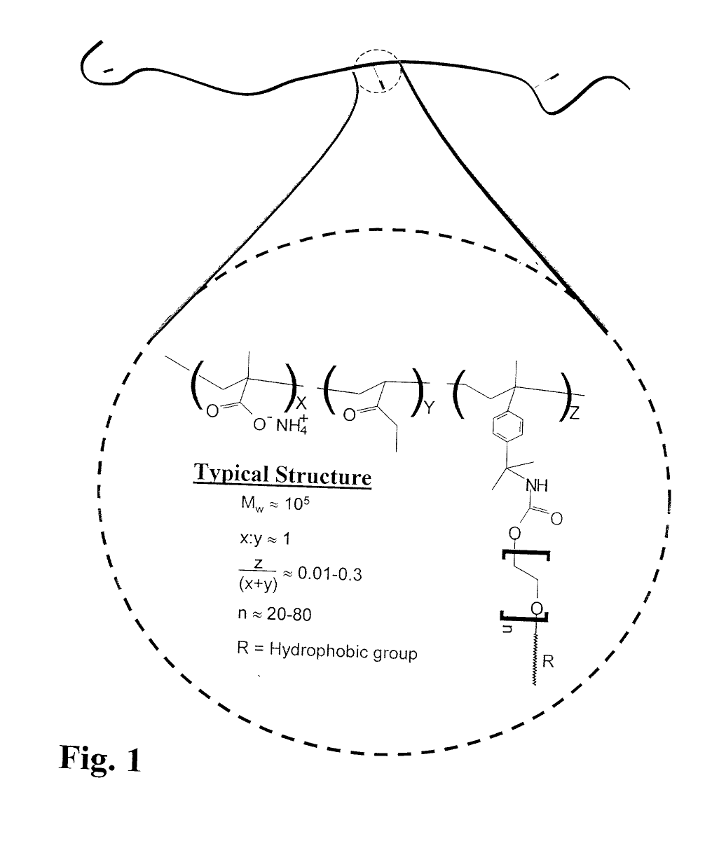

FIG. 1 depicts the chemical structure of an exemplary HASE polymer disclosed by Jenkins et al., supra. Generally, HASE polymers are amphiphilic. The backbone of the polymer chains in FIG. 1 contain carboxylic acid groups that are hydrophobic when in their protonated state resulting in aggregation into latex particles when HASE are synthesized. When a base, such as ammonia, is added to a HASE polymer, the acid groups are neutralized, making the backbone sufficiently hydrophilic for the latex particles to break apart. HASE polymers also include hydrophobic pendent groups which can be hydrocarbons, fluorocarbons, and silicon bearing. In aqueous media, the pendent hydrophobic groups associate into a network of micelle-like clusters and form a gel, thus increasing the viscosity of the polymer. See Winnik, et al., “Associative polymers in aqueous solution,” Current Opinion in Colloid and Interface Science 1997, 2, 424-436; and Horiuchi, et al., “Fluorescence Probe Studies of Hydrophobic Domains in a Model Hydrophobically Modified Alkali-Swellable Emulsion (HASE) Polymer With C₂₀H₄₁ Groups,” Langmuir 15, 1644-1650 (1999).

The dye may be any known in the art, such as those used in biological assays and standards and for calibrating instruments, such as spectrometers, multi-well plate readers, and imagers. The dye may be hydrophobic or hydrophilic. Water insoluble dyes, such as polycyclic aromatic hydrocarbons, generally solubilize in the micelles of the HASE polymers, while water soluble dyes, such as fluorescein, remain in the aqueous phase of the HASE polymers or become associated to the polymeric backbone of the HASE polymers. Suitable dyes include fluorescent dyes, such as fluorescein and derivatives thereof and the dye Cy3®, available from Amersham Pharmacia Biotech of Piscataway, N.J.

Dyes available in various degrees of hydrophobicity, such as those described in Haugland, supra, permit fine spectral tuning. For example, fluorescein is an ionic dye, but is also available with (hydrophobic) C₁₈ alkyl chains conjugated to it. The hydrophobic dye pyrene is available conjugated to an ionic group, such as, for example, the group—CH₂CH₂NH₃⁺Cl⁻. Selection of ordinary and modified dyes permits control of the dielectric constant of the medium in which the dye is present and, hence, spectral tuning in particular wavelength ranges, such as 5 to 50 nm.

Bioactive compounds are often assayed by determining the extent of binding of probes to receptors through the monitoring of fluorescence polarization. See Lakowicz, J. R., “Principles of Fluorescence Spectroscopy,” 2^{nd} ed., Kluwer Academic/Plenum Publishers, New York, 1999; Nasir et al., “Fluorescence Polarization: An Analytical tool for Immunoassay and Drug Discovery,” Com. Chem. High. T. Scr. 2, 177-190 (1999); Parker et al., “Development of High Throughput Screening Assays Using Fluorescence Polarization: Nuclear Receptor-Ligand-Binding and Kinase/Phosphatase Assays,” J. Biomol. Screen. 5, 77-88 (2000); and Banks et al., “Fluorescence Polarization Assays for High Throughput Screening of G Protein-Coupled Receptors,” ibid, 159-167 (2000). Therefore, it is desirable to have one or more standards in which their degree of fluorescence polarization can be controlled. The degree of polarized fluorescence of the standard of the present invention can be varied by selecting the appropriate dye. For example, (hydrophobic) C₁₆-tagged fluorescin binds to the micelles of the HASE polymers resulting in a highly polarized fluorescence emission. In contrast, untagged fluorescein is water soluble resulting in a mostly depolarized fluorescence emission. By combining two or more types of flourescent dyes, it is also possible to obtain intermediate states of fluorescence polarization.

Another method of determining the degree of binding of probes to receptors is by monitoring the degree to which they attain proximity. See Selvin, P. R., “Fluorescence Resonance Energy-Transfer,” Methods Enzymol. 246,300-334(1995). Typically, the probes and receptors are labeled with a suitable pair of donor and acceptor dyes which transfer energy when they are within close proximity (e.g., several nanometers). The standards of the present invention can mimic this behavior by including the donor and acceptor dyes in suitably hydrophobized forms. For example, (hydrophobic) di-C₁₈-labeled Cy3 and CyS dyes. These dyes bind to the micelles of the HASE polymers and transfer energy because the micelle sizes are in the nanometer scale range. Horiuchi, et al., supra. By adjusting the number of acceptor dyes per micelle, one controls the mean donor-acceptor separation distance, thereby simulating the extent of energy transfer and the conditions observed in real assays.

The standard may include additives known in the art, such as anti-foaming agents, buffers, pH adjusting agents, and solvents, such as those that control vapor pressure and surface tension (e.g., water and water-miscible organic solvents).

The standard may be prepared by mixing one or more viscosity changing polymers with at least one dye and gelling the mixture.

For example, for pH responsive polymers, the standard may be prepared by mixing one or more pH responsive polymers with at least one dye and increasing the pH of the resulting mixture until the mixture gels. The pH of the mixture may be increased by any method in the art, such as by reacting the mixture with a base (such as those described above). Another method of increasing the pH is by adding an alkaline agent to the mixture.

A preferred method of increasing the pH is by diffusing an alkaline gas, such as ammonia, through the mixture. This preserves the shape of the meniscus. The diffusion reaction causes the mixture to gel faster than a typical reaction affected by diffusion since the diffusion of hydronium ions is about an order of magnitude faster than all other species.

Once the gel is formed, its pH is preferably reduced to near neutral (e.g., between pH 6 and 8) to increase its chemical stability. For example, the gel may be placed in a chamber to reduce alkalinity (e.g., by removing excess ammonia present in the gel) while controlling its loss or gain of water content.

The liquid mixture (before it is viscosified into a gel) is preferably prepared outside a container and then is poured into it. The standard is preferably gelled in the container to be used in the instrument. Alternatively, the viscosity changing polymers and dye may be individually dispensed into a container and then mixed and gelled.

After the gel is formed on a container, a sheet of anti-reflective glass may be placed on the exposed surface of the standard to protect it.

The following example illustrates the invention without limitation. All parts and percentages are given by weight unless otherwise indicated.

This example incorporates the dye Cy3™ (Amersham Pharmacia Biotech, Piscataway, N.J., http://www.apbiotech.com/) in 384-well plates at 40 micro-liters per well. The dye's real concentration is adjusted to yield fluorescence intensities equaling that of a primary standard, 100 nM Cy3™ in TRIS/HCl buffer (pH 8), at 40 micro-liters per well, in the same plate format. The quantities given are for making one standard plate. It is understood that other dyes, or plate formats, can be easily substituted, with concentrations adjusted to yield intensities equal to the desired primary standards.

The processing involves preparing and dispensing the formulation fluid into 384-well plates (steps al-a5), triggering of the viscosifying gelation reaction with gaseous NH3 (steps b1-b3), adjusting the gel pH to near neutral (steps c1-c3), and sealing the plates with anti-reflective (AR) glass sheets (step d1). These steps are described in detail below.

(a1) Preparation of dispersion stock solution al: To 98.0 g of Polyphobe® TR116(Union Carbide Corp., Houston, Tex.) add2.0 g of antifoam TEGO2-89 (Goldschmidt Chemical Corp., Hopewell, Va.). Shake well.

(a2) Preparation of the CY3™ dye stock solution a2: Prepare about 100 mL of near 1 micro-molar solution of CY3™ in TRIS/HCl buffer (pH 8), according to the procedures specified by the manufacturer (Amersham Pharmacia Biotech, Piscataway, N.J.).

(a3) Preparation of the CY3™ dye stock solution a3: Prepare 300 g of about 80 nM Cy3 by adding 20.0 g of stock a2 to 80.0 g of water and 200.0 g of glycerol.

(a4) Preparation of the dispensing fluid a4: Prepare 50.0 g of a near 60 nM Cy3 dispersion by adding 37.5 g of Cy3 stock a3 to 12.5 g of dispersion stock al. Mix well.

(a5) Dispensing: Uniformly dispense stock a4 at 40 micro-liters per well, into the wells of a black 384-well plate, e.g. Costar™ (Corning Inc., Life Sciences, Acton, Mass.). Centrifuge the plate at 2000 RPM for 2 minutes.

(b1) Preparation of alkaline stock solution b1: Add 720 g of glycerol to 1000 g of 28 wt % ammonium hydroxide.

(b2) Preparation of alkaline chamber b2: Bubble a gentle stream of gaseous ammonia into the alkaline stock solution b1 taking the outflow gas into a shelved chamber that has space for about 5 to 10 well plates, well isolated from the atmosphere except for the entry and exit ports. Conduct the gas from the exit port to a hooded area.

(b3) Alkaline reaction: Let the properly humidified NH3 gas pass through chamber b2 for 1 hour. After the dispensing step a5, immediately transfer the plate into the chamber b2 and let stay for 48 hours at ambient temperatures (22±2° C.).

(c1) Preparation of acid stock solution c1: Add and mix 1200 g of 10 wt % sulfuric acid to 900 g of glycerol.

(c2) pH control chamber c2: Employ a chamber similar to that used in b2 and connect the exit port of the air-pump to the input port of chamber c2 via a bubbler containing sufficient quantity of the acid stock c1.

(c3) pH control: Let the atmosphere of chamber c2 be properly humidified by circulating its atmosphere through acid stock c1 for 1 hour. After the alkaline reaction step b3 is finished, remove the plate from chamber b2 and place it in chamber c2. Let stay for 72 hours at ambient temperatures (22±2° C.).

(d1) Sealing: Remove the plate from chamber c2 and cover it with a sheet of anti-reflective glass coated on both sides and cut to the shape of the plate top, to within ±0.5 mm (e.g., 0.048′ thick Invisiglass, Optical Coating Laboratory, Inc., Santa Rosa, Calif.). Use ½′ aluminum adhesive tape (e.g., # 425, 3M Co., St. Paul, Minn.) to seal the edges of plate and glass together. Use ½′ black adhesive tape (e.g., nonfluorescent electrical tape) to cover the reflective areas of the aluminum tape.

In accordance with the present invention, it is also contemplated that fluid solutions of dye-containing compositions be dispensed into cuvettes, micro-well plates, or other desirable containers, to be later viscosified, by lowering of temperature, into mechanically stable clear gels. Preferably, the fluid has a viscosity higher than 100 Poise (gram sec^{31 1} cm^{31 1}) when the temperature is between 30° C. to 20° C., while viscosity is lowered to less than 10 Poise when heated anywhere between 30° C. to 70° C. Preferably, the active viscosifying agent is any member of thermoreversible hydrogels.

All patents, publications, applications, and test methods mentioned above are hereby incorporated by reference. Many variations of the present matter will suggest themselves to those skilled in the art in light of the above detailed description. All such obvious variations are within the patented scope of the appended claims.

We claim:

1. A standard for calibrating an instrument comprising:
(a) one or more viscosity changing polymers; and
(b) at least one dye.

2. The standard of claim 1, wherein the viscosity changing polymer is a pH responsive polymer, a temperature responsive polymer, or any mixture thereof.

3. The standard of claim 2, wherein the viscosity changing polymer is a pH responsive polymer.

4. The standard of claim 3, wherein the pH responsive polymer is a liquid at a pH of less than about 4.5.

5. The standard of claim 3, wherein the pH responsive polymer is a hydrophobically-modified alkali-swellable emulsion polymer.

6. The standard of claim 5, wherein the hydrophobically-modified alkali-swellable emulsion is an acrylic carboxylate emulsion polymer.

7. The standard of claim 5, wherein the hydrophobically-modified alkali-swellable emulsion is an alkali-swellable emulsion urethane-modified emulsion polymer.

8. The standard of claim 1, wherein the viscosity changing polymer has a viscosity of at least about 10,000 cP.

9. The standard of claim 8, wherein the viscosity changing polymer has a viscosity of at least about 100,000 cP.

10. The standard of claim 1, wherein the viscosity changing polymer is transparent to light at a wavelength ranging from about 300 to about 1,000 nm.

11. The standard of claim 1, wherein the dye is a fluorescent dye.

12. The standard of claim 1, wherein the instrument is a spectrometer, multiwell plate reader, or imager.

13. A container for calibrating a spectrometer comprising:
(a) a container; and
(b) a standard of claim 1 in or on the container.

14. The container of claim 13, wherein the container is a plate.

15. The plate of claim 14, wherein the plate is a micro-well plate and the standard is in at least one micro-well of the plate.

16. The container of claim 13, wherein the container is a cuvette.

17. A process for preparing a standard comprising the steps of:
(a) mixing one or more viscosity changing polymers and at least one dye; and
(b) gelling the mixture.

18. A process for preparing a container for calibrating an instrument comprising the steps of:
(a) dispensing one or more viscosity changing polymers and at least one dye into a container to form a mixture; and
(b) gelling the mixture.

19. The process of claim 18, wherein step (a) comprises the steps of:
(i) mixing the viscosity changing polymers and the dye; and
(ii) dispensing the mixture into the container.

20. The process of claim 18, wherein the viscosity of the viscosity changing polymer being dispensed ranges from about 1 to about 1,000 cP.

21. The process of claim 18, wherein the viscosity changing polymer is a pH responsive polymer.

22. The process of claim 21, wherein step (b) comprises increasing the pH of the mixture sufficiently to gel the mixture.

23. The process of claim 22, wherein the mixture in step (a) has a pH of less than about 4.5 and step (b) comprises increasing the pH to at least about 5.

24. The process of claim 22, wherein step (b) comprises diffusing an alkaline gas through the mixture.

25. The process of claim 24, wherein the alkaline gas is ammonia gas.

26. The process of claim 22, further comprising the step of:
(c) neutralizing the gel formed in step (b) to a pH of from about 6 to about 8.

27. The process of claim 18, wherein the viscosity of the viscosity changing polymer in the gel in step (b) is at about 10,000 cP.

28. A method for calibrating an instrument comprising the step of calibrating the instrument with the standard of claim 1.

29. The method of claim 28, wherein the instrument is a spectrometer, multiwell plate reader, or imager.
